(19) United States
(12) Patent Application Publication
(10) Pub. No.: US 2014/0378406 A1
(43) Pub. Date: Dec. 25, 2014

(54) SILVER BASED GELS FOR ANTIMICROBIAL APPLICATIONS

(71) Applicant: The University of Akron, Akron, OH (US)

(72) Inventors: Thomas Leeper, Fairlawn, OH (US); Matthew Panzner, Akron, OH (US); Wiley Youngs, Akron, OH (US); Mitch Kovacs, Medina, OH (US)

(21) Appl. No.: 14/368,981

(22) PCT Filed: Mar. 1, 2013
(86) PCT No.: PCT/US13/28494

§ 371 Date: Jun. 26, 2014

Related U.S. Application Data
(60) Provisional application No. 61/605,508, filed on Mar. 1, 2012.

(51) Int. Cl. A61K 31/555; A61K 31/7036; A61K 31/397; A61K 31/192; A61K 31/198; A61K 9/06

(52) U.S. Cl. CPC A61K 31/555; A61K 31/198; A61K 9/06; A61K 31/397; A61K 31/192; A61K 31/7036
USPC 514/40; 514/186; 514/184

(57) ABSTRACT

A pharmaceutical composition is prepared by mixing a cysteine-based monomer unit and a silver carbene complex. The cysteine-based monomer may be glutathione and the silver carbine complex may be provided according to particular formulae. The mixtures form gels and the gels may further include additional drug components.

### CROSS-REFERENCE TO RELATED APPLICATIONS

The present application claims the benefit of U.S. Provisional Patent No. 61/605,508 filled Mar. 1, 2012.

### FIELD OF THE INVENTION

This invention resides generally in the field of pharmaceutical compositions for the treatment and prevention of microbial infections. In one embodiment this invention relates to silver based gels with antimicrobial functionality. In other embodiments this invention relates to silver based gels made by the combination of silver carbene complexes and cysteine-based monomer units.

### BACKGROUND OF THE INVENTION

A leading cause of human disease and mortality is microbial infection. The over prescription and aggressive dosing of clinical antibiotics has led to an epidemic of MDR bacterial strains. One classic example is the resistance of certain strains of Staphylococcus aureus to penicillin and its derivatives (e.g. MRSA). This resistance arose only a few years after the introduction of penicillin for civilian use. In recent decades this phenomenon has been accelerated with resistance to each new class of organic antibiotic occurring quickly after its introduction into clinical use. In the 1960s mounting antibiotic resistance prompted clinicians to return to silver nitrate to treat burn wounds, a practice that lead to the advent of the silver sulfadiazine cream (Silvadene) in 1968. Today, Silvadene is used in virtually every burn clinic in the country, and despite its extensive use, resistance to silver has remained exceptionally rare.

However, because of its formulation, Silvadene has some undesirable limitations. Silvadene is a white opaque material and must be removed in order to observe a wound which results in patient discomfort and pain. The non-silver based component of Silvadene, sulfadiazine, is a sulfonamide antibiotic. Sulfonamides have been shown to elicit nephrotoxicity and metabolic acidosis, most likely as a result of inhibition of the active site zinc in carbonic anhydrase (CA) isoforms.

Therefore there exists a need in the art for a silver based antimicrobial imbedded in a clear biologically based gel/polymer matrix through which the wound or burn could be easily observed.

### SUMMARY OF THE INVENTION

A first embodiment of this invention provides a pharmaceutical composition prepared by mixing a cysteine-based monomer unit and a silver carbene complex.

A second embodiment provides a pharmaceutical composition as in the first embodiment, wherein the cysteine-based monomer unit is selected from the group consisting of units of cysteine, cysteine derivatives, and cysteine containing peptides according to the following structure:

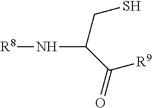

wherein R8 is selected from the group consisting of a hydrogen, an amino acid and an acetyl group, and wherein R9 is selected from the group consisting of an amino acid, a hydroxyl group and an ester.

A third embodiment provides a pharmaceutical composition as in either the first or second embodiment, wherein the cysteine containing peptide is glutathione.

A fourth embodiment provides a pharmaceutical composition as in any of the first through third embodiments, wherein the silver carbene complex is selected from the group consisting of:

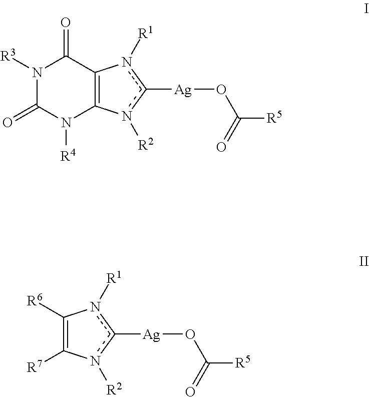

wherein R1, R2, R3, R4, R5, R6 and R7 are selected from the group consisting of hydrogen; methyl; hydroxy; C1 to C12 alkyl; C1 to C12 substituted alkyl; C3 to C12 cycloalkyl; C3 to C12 substituted cycloalkyl; C2 to C12 alkenyl; C3 to C12 cycloalkenyl; C3 to C12 substituted cycloalkenyl; C2 to C12 alkynyl; C6 to C12 aryl; C5 to C12 substituted aryl; C6 to C12 arylalkyl; C6 to C12 alkylaryl; C3 to C12 heterocyclic; C3 to C12 substituted heterocyclic; C1 to C12 alkoxy; C1 to C12 alcohols; C1 to C12 carboxy; biphenyl; C1 to C6 alkyl biphenyl; C2 to C6 alkenyl biphenyl; and C2 to C6 alkynyl biphenyl; wherein R5 is further selected from a derivative of an additional drug component selected from fluoroquinolones, penicillins, tetracyclines, antibacterial compounds, derivatives of antibacterial compounds, ibuprofen, anti-inflammatory compounds, derivatives of anti-inflammatory compounds, anti-fungal compounds, derivatives of anti-fungal compounds, steroids and derivatives of steroids; and wherein R6 and R7 are further selected from halogens.

A fifth embodiment provides a pharmaceutical composition as in any of the first through fourth embodiments, wherein the silver carbene complex is selected from the group consisting of:

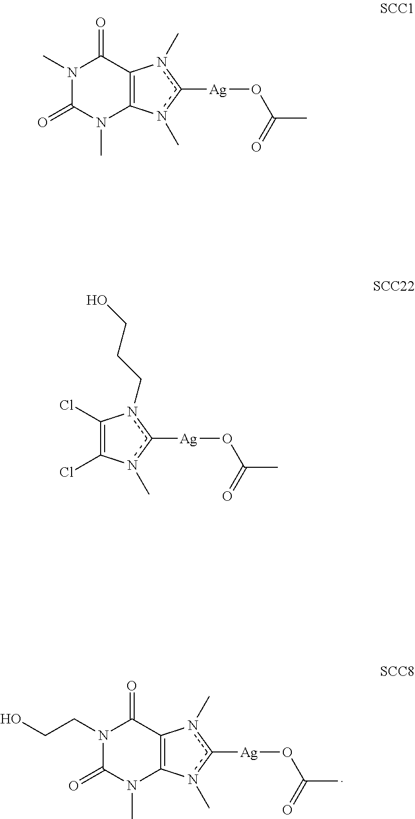

A sixth embodiment provides a pharmaceutical composition as in any of the first through fifth embodiments, wherein the cysteine-based monomer unit is glutathione and the silver carbene complex is SCC22.

A seventh embodiment provides a pharmaceutical composition as in any of the first through sixth embodiments, wherein the glutathione and SCC22 are combined in a molar ratio of from 1:1 to 1:2.

An eighth embodiment provides a pharmaceutical composition as in any of the first through seventh embodiments, wherein the cysteine-based monomer unit is glutathione and the silver carbene complex is SCC1.

A ninth embodiment provides a pharmaceutical composition as in any of the first through eighth embodiments, wherein the glutathione and SCC1 are combined in a molar ratio of from 1:1 to 1:2.

A tenth embodiment provides a pharmaceutical composition as in any of the first through ninth embodiments, wherein the cysteine-based monomer unit is glutathione and the silver carbene complex is SCC8.

An eleventh embodiment provides a pharmaceutical composition as in any of the first through tenth embodiments, wherein the glutathione and SCC8 are combined in a molar ratio of from 1:1 to 1:2.

A twelfth embodiment provides a pharmaceutical composition as in any of the first through eleventh embodiments, wherein the pharmaceutical composition is further prepared by mixing an additional drug component with one or both of the cysteine-based monomer unit and silver carbene complex, the additional drug component selected from the group consisting of quinolone compounds and derivatives thereof; fluoroquinolone compounds and derivatives thereof; penicillin compounds and derivatives thereof; aminoglycoside compounds and derivatives thereof; cephalosporins compounds and derivatives thereof; glycopeptides and derivatives thereof; sulfonamides and derivatives thereof; tetracycline and derivatives thereof; steroids and derivatives thereof; anti-inflammatory compounds and derivatives thereof; analgesic compounds and derivatives thereof; anti-fungal compounds and derivatives thereof; anti-bacterial compounds and derivatives thereof; and tissue growth factors

A thirteenth embodiment provides a pharmaceutical composition as in any of the first through twelfth embodiments, wherein the cysteine-based monomer unit is glutathione, the silver carbene complex is SCC22 and the additional drug species is carbenicillin disodium salt.

A fourteenth embodiment provides a pharmaceutical composition as in any of the first through thirteenth embodiments, wherein the glutathione, SCC22, and the carbenicillin disodium salt are combined in a molar ratio of from 1:1:0.2 to 1:2:1.

A fifteenth embodiment provides a pharmaceutical composition as in any of the first through fourteenth embodiments, wherein the cysteine-based monomer unit is glutathione, the silver carbene complex is SCC22 and the additional drug species is ibuprofen sodium salt.

A sixteenth embodiment provides a pharmaceutical composition as in any of the first through fifteenth embodiments, wherein the glutathione, SCC22, and the ibuprofen sodium salt are combined in a molar ratio of from 1:1:0.2 to 1:2:1.

A seventeenth embodiment provides a pharmaceutical composition as in any of the first through eighteenth embodiments, wherein the cysteine-based monomer unit and a silver carbene complex are mixed by creating liquid stocks of each component and then mixing those liquid stocks to form a gel.

An eighteenth embodiment provides a pharmaceutical composition as in any of the first through seventeenth embodiments, wherein the cysteine-based monomer unit and a silver carbene complex are mixed by measuring out the cysteine-based monomer unit and silver carbene complex in dry powder form and then adding water to produce a final mixture to form a gel.

### DETAILED DESCRIPTION OF ILLUSTRATIVE EMBODIMENTS

The present invention provides a pharmaceutical composition for the treatment and prevention of microbial infections made by the combination of silver based carbene complexes and cysteine-based monomer units.

The silver carbene complex may be selected from

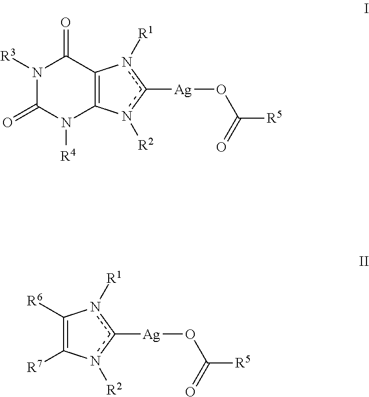

where R¹, R², R³, R⁴, R⁵, R⁶ and R⁷ are each independently selected from hydrogen, methyl, hydroxyl, C₁ to C₁₂ alkyl, C₁ to C₁₂ substituted alkyl, C₃ to C₁₂ cycloalkyl, C₃ to C₁₂ substituted cycloalkyl, C₂ to C₁₂ alkenyl, C₃ to C₁₂ cycloalkenyl, C₃ to C₁₂ substituted cycloalkenyl, C₂ to C₁₂ alkynyl, C₆ to C₁₂ aryl, C₅ to C₁₂ substituted aryl, C₆ to C₁₂ arylalkyl, C₆ to C₁₂ alkylaryl, C₃ to C₁₂ heterocyclic, C₃ to C₁₂ substituted heterocyclic, C₁ to C₁₂ alkoxy, C₁ to C₁₂ alcohols, C₁ to C₁₂ carboxy, biphenyl, C₁ to C₆ alkyl biphenyl, C₂ to C₆ alkenyl biphenyl, and C₂ to C₆ alkynyl biphenyl.

In some embodiments, R⁵ is a derivative of an additional drug component selected from fluoroquinolones, penicillins, tetracyclines, antibacterial compounds, derivatives of antibacterial compounds, ibuprofen, anti-inflammatory compounds, derivatives of anti-inflammatory compounds, anti-fungal compounds, derivatives of anti-fungal compounds, steroids and derivatives of steroids. By “derivative of” it is to be understood that the additional drug components listed include carboxylic acid groups and are bound to the silver by means of that carboxylic acid group, as represented by the —COO group in formulas I and II above. The method of binding additional drug components to silver is known, as for example in US2007/0021401, US2008/0267867, US2010/0049617, US2010/0204193, US2011/0086830, and US2011/0306583.

In some embodiments, R⁶ and R⁷ are further selected from halogens.

The cysteine-based monomer unit may be selected from units of cysteine, cysteine derivatives, and cysteine containing peptides per the following structure:

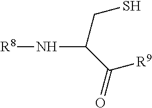

wherein R⁸ is selected from hydrogen, an amino acid(s), and an acetyl group, and R⁹ is selected from an amino acid(s), a hydroxyl group, and an ester.

In some embodiments, the cysteine-based monomer unit and the silver carbene complex are mixed by first creating liquid stocks of each component and then mixing those liquid stocks. In other embodiments, the cysteine-based monomer unit and the silver carbene complex are mixed by measuring out suitable amounts of the cysteine-based monomer unit and silver carbene complex in dry powder form and then adding water to produce a final mixture.

In some embodiments, the cysteine-based monomer unit and the silver carbene complex are mixed at molar ratios of from 1:1 to 1:2 (cysteine-based monomer unit:silver carbene complex) by mixing liquid stocks of each component to final concentrations ranging from 5 mM to 50 mM. In addition bulk mixtures may also be prepared by measuring out suitable amounts of the cysteine-based monomer unit and silver carbene complex in dry powder form to mix at molar ratios of from 1:1 to 1:2 and then adding water to produce a final solution of from 5 mM to 50 mM of each component. These general mix ratios apply to all embodiments herein, though more particular mix ratios are provided below with respect to particular embodiments.

In a particular embodiment, the cysteine-based monomer unit is glutathione. In some embodiments the silver carbene complex is selected from SCC22, SCC1, and SCC8.

In some embodiments, the cysteine-based monomer unit is glutathione and the silver carbene complex is SCC22. These are mixed as shown below:

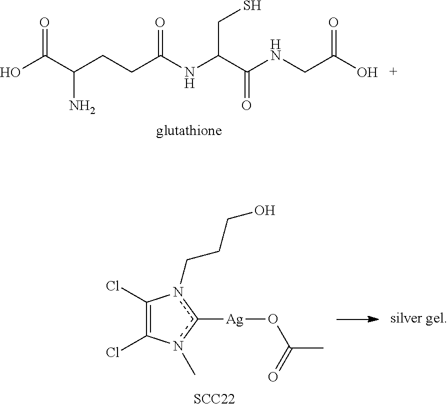

In a particular embodiment the glutathione and SCC22 can be mixed at a molar ratio of from 1:1 (glutathione: SCC22) to 1:2, for example, at a final concentration of 5 mM of glutathione to 5 mM of SCC22 or 30 mM of glutathione to 40 mM of SCC22. In a particular embodiment glutathione is mixed with SCC22 to afford a 1:1.25 molar ratio with a final concentration of 20 mM of glutathione and 25 mM of SCC22.

In some embodiments, the cysteine-based monomer unit is glutathione and the silver carbene complex is SCC1. These are mixed as shown below:

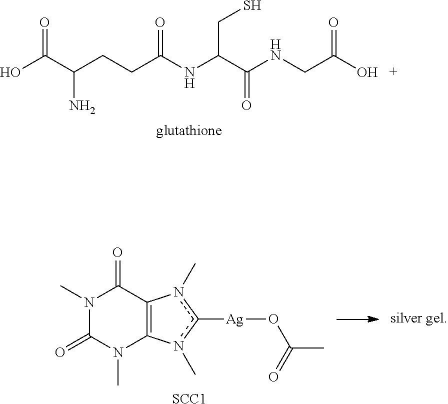

In a particular embodiment the glutathione and SCC1 can be mixed at a molar ratio of from 1:1 (glutathione:SCC1) to 1:2, for example, at a final concentration of 5 mM of glutathione to 5 mM of SCC1 or 25 mM of glutathione to 25 mM of SCC1. In a particular embodiment glutathione is mixed with SCC1 to afford a 1:1.67 molar ratio with a final concentration of 15 mM of glutathione and 25 mM of SCC1.

In yet some other embodiments, the cysteine-based monomer unit is glutathione and the silver carbene complex is SCC8. These are mixed as shown below:

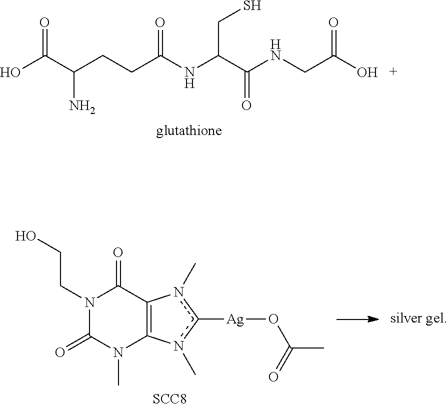

The glutathione and SCC8 can be mixed at molar ratios as described above in paragraph [0013]. In a particular embodiment the glutathione and SCC8 can be mixed at a molar ratio of from 1:1 (glutathione:SCC8) to 1:2, for example, at a final concentration of 5 mM of glutathione to 5 mM of SCC8 or 30 mM of glutathione to 40 mM of SCC8. In a particular embodiment glutathione is mixed with SCC8 to afford a 1:1.25 molar ratio with a final concentration of 20 mM of glutathione and 25 mM of SCC8.

Notably, the pharmaceutical compositions of the present invention can be made by simply mixing the chosen cysteine-based monomer unit and the chosen silver carbene complex at room temperature and pressure. Upon mixing, a gel forms over time on the order of about 5 minutes to 3 hours depending on the absolute concentrations of the two species.

In particular embodiments the pharmaceutical compositions of the present invention can be made by adding dry powder versions of the chosen cysteine-based monomer unit and chosen silver carbene complex to water. In a specific embodiment, a separate liquid stock of the chosen cysteine-based monomer unit and a separate liquid stock of the chosen silver carbene complex are first created, and these stocks are mixed together to form the pharmaceutical composition of the present invention.

The pharmaceutical composition in the form of a gel is stable in the presence of ambient light and will retain its clarity, viscosity and antimicrobial properties for months. In particular embodiments of the present invention in which the chosen cysteine-based monomer unit is glutathione, the pharmaceutical composition would not be expected to elicit negative side effects when used topically because glutathione is a well-established nutritional supplement and cosmetic antioxidant.

In other embodiments, the gels herein are amenable to the incorporation of one or more additional drug components into their formulations without the loss of the aforementioned properties. These additional drug components can be included in the mixture of cysteine-based monomer and silver carbine complex to be physically present in the gel that forms. They may be incorporated into one or more of the aforementioned liquid stocks or may be part of the dry bulk mixture of components to which water is then added to create the gel. They might also be formed as their own liquid stock.

These additional drug components are selected from the group consisting of quinolone compounds and derivatives thereof; fluoroquinolone compounds and derivatives thereof; penicillin compounds and derivatives thereof; aminoglycoside compounds and derivatives thereof; cephalosporins compounds and derivatives thereof; glycopeptides and derivatives thereof; sulfonamides and derivatives thereof; tetracycline and derivatives thereof; steroids and derivatives thereof; anti-inflammatory compounds and derivatives thereof; analgesic compounds and derivatives thereof; anti-fungal compounds and derivatives thereof; and tissue growth factors.

Thus, the gels herein can include (a) no additional drug components, (b) an R⁵ group derived from an additional drug component as described above, (c) an additional drug component physically present in the gel that is formed or (d) both an R⁵ group derived from an additional drug component and an additional drug component physically present in the gel that is formed. Thus, the additional drug components listed here can be added alone or in addition to any additional drug components at —OOCR⁵.

In some embodiments, the cysteine-based monomer unit, silver carbene complex, and additional drug component are prepared at molar ratios of from 1:1:0.2 to 1:2:1 (cysteine-based monomer unit:silver carbene complex:additional drug component) by mixing liquid stocks of each component to final concentrations ranging from 5 mM to 50 mM. In addition bulk mixtures may also be prepared by measuring out suitable amounts of the cysteine-based monomer unit, silver carbene complex, and additional drug component in dry powder form to mix at molar ratios of from 1:1:0.2 to 1:2:1 and then water is added to produce a final solution of from 5 mM to 50 mM of each component. These general mix ratios apply to all embodiments herein, though more particular mix ratios are provided below with respect to particular embodiments.

In some embodiments an ibuprofen sodium salt is added to the ingredients such that the resultant gel also serves to deliver ibuprofen to a wound or infection. They are combined as shown below:

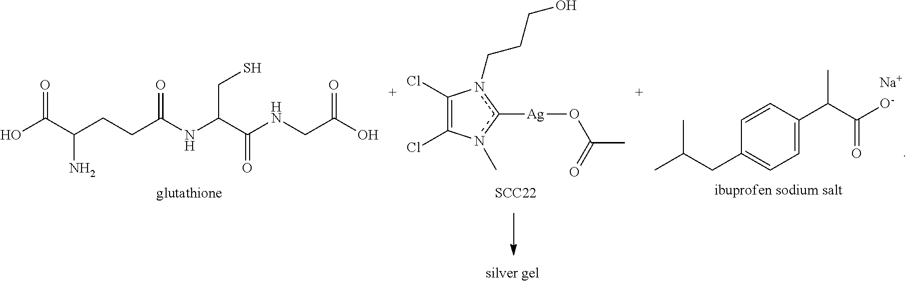

In a particular embodiment, the glutathione, SCC22, and ibuprofen sodium salt is mixed at molar ratios of from 1:1:0.2 (glutathione:SCC22:ibuprofen sodium salt) to 1:2:1, for example, at a final concentration of 20 mM of glutathione, to 22.5 mM of SCC22, and to 5 mM of ibuprofen salt or 20 mM of glutathione, to 25 mM of SCC22, and to 5 mM of ibuprofen salt. In a particular embodiment glutathione is mixed with SCC22 and ibuprofen sodium salt to afford a molar ratio of 1:1.125:0.25 with a final concentration of 20 mM of glutathione, to 22.5 mM of SCC22, and 5 mM of ibuprofen sodium salt.

In some embodiments, a carbenicillin disodium salt is added to the ingredients such that the resultant gel also serves to deliver carbenicillin to a wound or infection. They are combined as shown below:

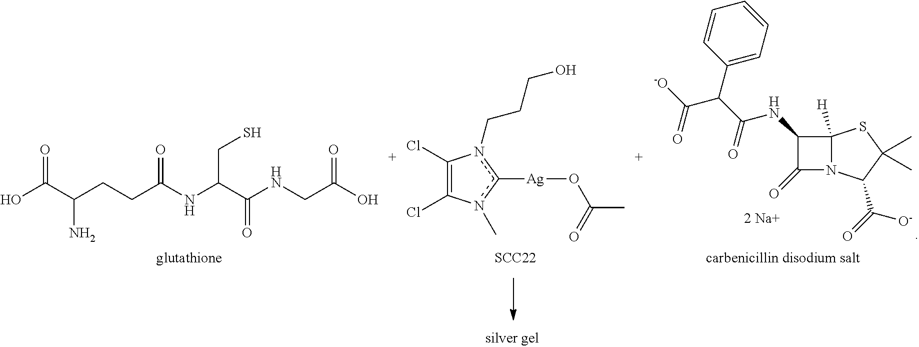

In a particular embodiment, the glutathione, SCC22, and carbenicillin disodium salt is mixed at molar ratios of from 1:1:0.2 (glutathione:SCC22:carbenicillin disodium salt) to 1:2:1, for example, at a final concentration of 22.5 mM of glutathione, to 22.5 mM of SCC22, and to 5 mM of carbenicillin disodium salt or 20 mM of glutathione, to 25 mM of SCC22, and to 5 mM of carbenicillin disodium salt. In a particular embodiment glutathione is mixed with SCC22 and carbenicillin disodium salt to afford a molar ratio of 1:1:0.22 with a final concentration of 22.5 mM of glutathione, 22.5 mM of SCC22, and 5 mM of carbenicillin disodium salt.

Gels of this invention test well as to minimal inhibitory concentration (MIC) on bacterial species. They also have been observed to qualitatively exhibit bacterial growth inhibition. While testing the bacterial growth inhibition, four formulations of glutathione and SCC22 were tested. The molar ratios of the four formulations (glutathione:SCC22) were 1:1.25 with a final concentration of 20 mM glutathione and 25 mM SCC22, 1:1 with a final concentration of 25 mM glutathione and 25 mM SCC22, 1:1.2 with a final concentration of 25 mM glutathione and 30 mM SCC22, and 1:1 with a final concentration of 30 mM glutathione and 30 mM SCC22. Bacterial culture plates were first inoculated with a pair of bacterial strains (E. coli and P. aeruginosa) and subsequently a line of a pharmaceutical composition gel in accordance with this invention was extruded onto the surface of the plate followed by an 18 hour incubation period. During this time, a bacterial lawn grew in region greater than ˜2 cm from the line, e.g. from 50% to 90% of the total plate surface. The shape of the zone of inhibition did not perfectly match the shape of the extruded line indicating that the hydrogel releases bacteriocydal components capable of diffusion into the surrounding media. The significance of this result suggests that this hydrogel will have favorable properties for drug release into tissues during wound management. The size of the zone also correlated with MICs for these bacteria suggesting that this affect may be dose responsive.

In light of the foregoing, it should be appreciated that the present invention significantly advances the art by providing a pharmaceutical composition for the treatment and prevention of microbial infections that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

### Example 1

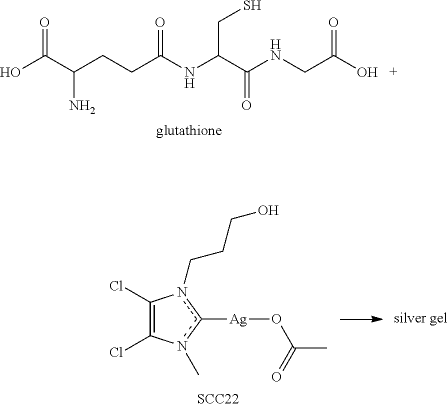

The combination of aqueous solutions of 50 mM glutathione and 50 mM silver carbene complex SCC22 at ambient conditions in a 1:1.25 ratio (final concentration 20 mM glutathione and 25 mM SCC22) results in a colorless hydrogel material. The gel was tested to determine its minimal inhibitory concentration (MIC) on three bacterial species including Pseudomonas aeruginosa, Staphylococcus aureus, and Escherichia coli using procedures published by the Clinical and Laboratory Standards Institute.

### MIC Results 4:5 Glutathione SCC22 Gel

| | MIC (g/mL) | | |
|---|---|---|---|
| Compound | P. aeruginosa | S. aureus | E. coli |
| Tobramycin* | 4 | 4 | 4 |
| SCC22* | 4 | 4 | 4 |
| GSH22 Gel | 4 | 4 | 4 |

*Indicates control antibiotic.

### Example 2

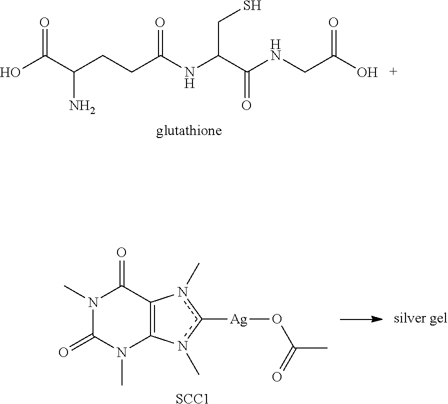

The combination of aqueous solutions of 50 mM glutathione and 50 mM silver carbene complex SCC1 at ambient conditions in a 1:1.67 ratio (final concentration 15 mM glutathione and 25 mM SCC1) results in a colorless hydrogel material. The gel was tested to determine its minimal inhibitory concentration (MIC) on three bacterial species including Pseudomonas aeruginosa, Staphylococcus aureus, and Escherichia coli using procedures published by the Clinical and Laboratory Standards Institute.

### MIC Results 3:5 Glutathione SCC1 Gel

| | MIC (g/mL) | | |
|---|---|---|---|
| Compound | P. aeruginosa | S. aureus | E. coli |
| Tobramycin* | 4 | 4 | 4 |
| SCC1* | 4 | 4 | 4 |
| GSH1 Gel | 16 | 16 | 16 |

*Indicates control antibiotic.

### Example 3

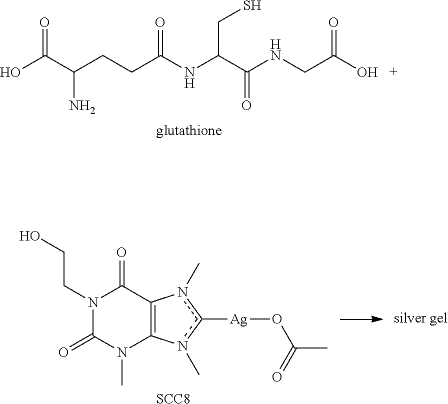

The combination of aqueous solutions of 50 mM glutathione and 50 mM silver carbene complex SCC8 at ambient conditions in a 1:1.25 ratio (final concentration 20 mM glutathione and 25 mM SCC8) results in a colorless hydrogel material (GSH8). The gel was tested to determine its minimal inhibitory concentration (MIC) on three bacterial species including Pseudomonas aeruginosa, Staphylococcus aureus, and Escherichia coli using procedures published by the Clinical and Laboratory Standards Institute.

### MIC Results 4:5 Glutathione SCC8 Gel

| | MIC (g/mL) | | |
|---|---|---|---|
| Compound | P. aeruginosa | S. aureus | E. coli |
| Tobramycin | 4 | 4 | 4 |
| SCC8 | 4 | 4 | 4 |
| GSH8 Gel | 4 | — | 4 |

* Indicates control antibiotic.

### Example 4

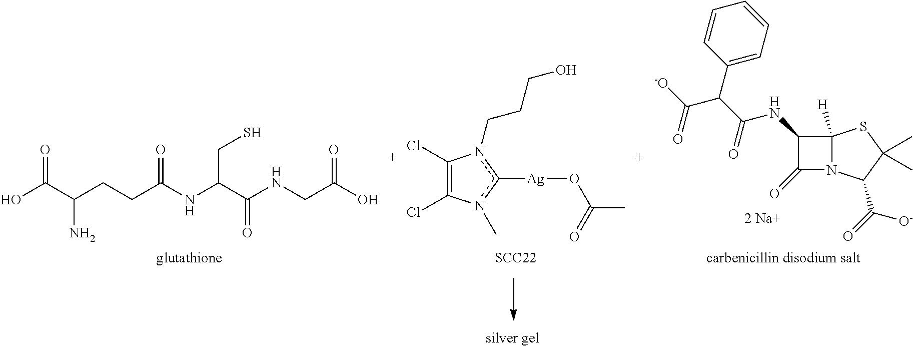

The combination of aqueous solutions of 50 mM glutathione, 50 mM silver carbene complex SCC22, and 50 mM carbenicillin disodium salt at ambient conditions in a 1:1:0.22 ratio (final concentration 22.5 mM glutathione, 22.5 mM SCC1, and 5 mM carbenicillin disodium salt) results in a colorless hydrogel material.

### Example 5

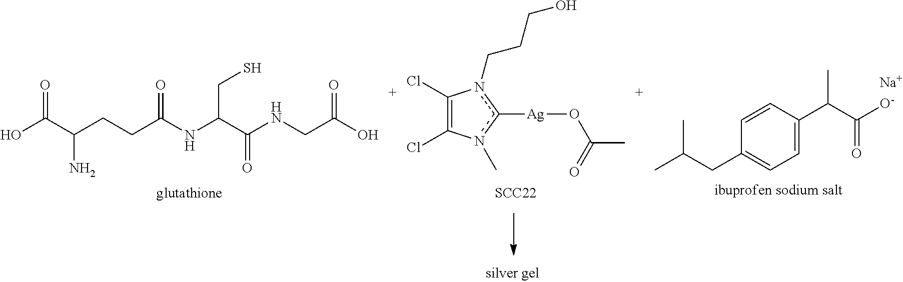

The combination of aqueous solutions of 50 mM glutathione, 50 mM silver carbene complex SCC22, and 50 mM ibuprofen sodium salt at ambient conditions in a 1:1.125:0.25 ratio (final concentration 20 mM glutathione, 22.5 mM SCC1, and 5 mM ibuprofen sodium salt) results in a colorless hydrogel material.

1. A pharmaceutical gel prepared by mixing glutathione and a silver carbene complex.

2. (canceled)

3. (canceled)

4. The pharmaceutical composition of claim 1, wherein the silver carbene complex is selected from the group consisting of: 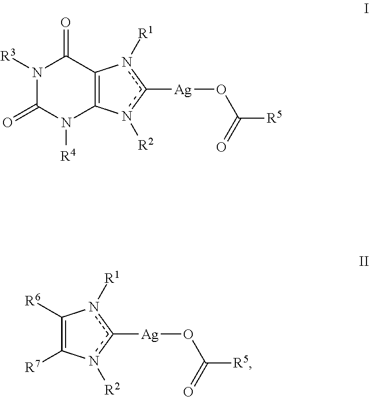
wherein R¹, R², R³, R⁴, R⁵, R⁶ and R⁷ are selected from the group consisting of hydrogen; methyl; hydroxy; C₁ to C₁₂ alkyl; C₁ to C₁₂ substituted alkyl; C₃ to C₁₂ cycloalkyl; C₃ to C₁₂ substituted cycloalkyl; C₂ to C₁₂ alkenyl; C₃ to C₁₂ cycloalkenyl; C₃ to C₁₂ substituted cycloalkenyl; C₂ to C₁₂ alkynyl; C₆ to C₁₂ aryl; C₅ to C₁₂ substituted aryl; C₆ to C₁₂ arylalkyl; C₆ to C₁₂ alkylaryl; C₃ to C₁₂ heterocyclic; C₃ to C₁₂ substituted heterocyclic; C₁ to C₁₂ alkoxy; C₁ to C₁₂ alcohols; C₁ to C₁₂ carboxy; biphenyl; C₁ to C₆ alkyl biphenyl; C₂ to C₆ alkenyl biphenyl; and C₂ to C₆ alkynyl biphenyl; wherein R⁵ is further selected from a derivative of an additional drug component selected from fluoroquinolones, penicillins, tetracyclines, antibacterial compounds, derivatives of antibacterial compounds, ibuprofen, anti-inflammatory compounds, derivatives of anti-inflammatory compounds, anti-fungal compounds, derivatives of anti-fungal compounds, steroids and derivatives of steroids; and wherein R⁶ and R⁷ are further selected from halogens.

5. The pharmaceutical composition of claim 4, wherein the silver carbene complex is selected from the group consisting of:

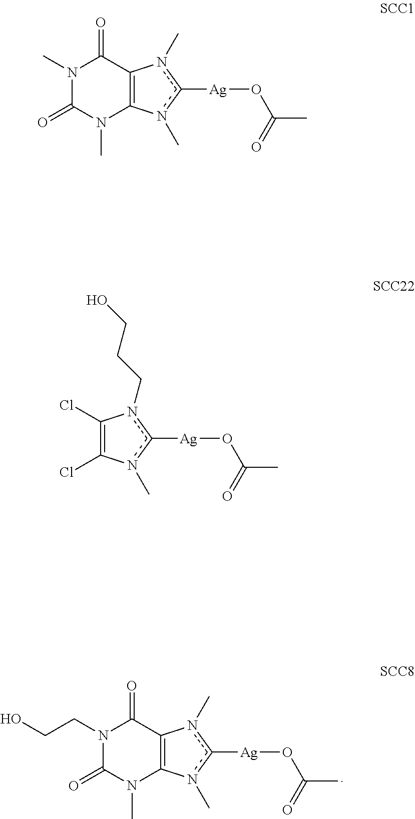

6. The pharmaceutical composition of claim 1, wherein the silver carbene complex is SCC22.

7. The pharmaceutical composition of claim 6, wherein the glutathione and SCC22 are combined in a molar ratio of from 1:1 to 1:2.

8. The pharmaceutical composition of claim 1, wherein the silver carbene complex is SCC1.

9. The pharmaceutical composition of claim 8, wherein the glutathione and SCC1 are combined in a molar ratio of from 1:1 to 1:2.

10. The pharmaceutical composition of claim 1, wherein the silver carbene complex is SCC8.

11. The pharmaceutical composition of claim 10, wherein the glutathione and SCC8 are combined in a molar ratio of from 1:1 to 1:2.

12. The pharmaceutical composition of claim 1, further prepared by mixing an additional drug component with one or both of the gluatathione and silver carbene complex, the additional drug component selected from the group consisting of quinolone compounds and derivatives thereof;
fluoroquinolone compounds and derivatives thereof; penicillin compounds and derivatives thereof; aminoglycoside compounds and derivatives thereof; cephalosporins compounds and derivatives thereof; glycopeptides and derivatives thereof; sulfonamides and derivatives thereof; tetracycline and derivatives thereof; steroids and derivatives thereof; anti-inflammatory compounds and derivatives thereof; analgesic compounds and derivatives thereof; anti-fungal compounds and derivatives thereof; anti-bacterial compounds and derivatives thereof; and tissue growth factors

13. The pharmaceutical composition of claim 12, wherein the the silver carbene complex is SCC22 and the additional drug species is carbenicillin disodium salt.

14. The pharmaceutical composition of claim 13, wherein the glutathione, SCC22, and the carbenicillin disodium salt are combined in a molar ratio of from 1:1:0.2 to 1:2:1.

15. The pharmaceutical composition of claim 12, wherein the silver carbene complex is SCC22 and the additional drug species is ibuprofen sodium salt.

16. The pharmaceutical composition of claim 15, wherein the glutathione, SCC22, and the ibuprofen sodium salt are combined in a molar ratio of from 1:1:0.2 to 1:2:1.

17. The pharmaceutical composition of claim 1, wherein the gluatathione and the silver carbene complex are mixed by creating liquid stocks of each component and then mixing those liquid stocks to form a gel.

18. The pharmaceutical composition of claim 1, wherein the gluatathione and the silver carbene complex are mixed by measuring out the gluatathione and the silver carbene complex in dry powder form and then adding water to produce a final mixture to form a gel.

19. A method of forming a pharmaceutical gel comprising the steps of mixing together glutathione and a silver carbene complex and allowing the pharmaceutical gel to form over time.

20. The method of forming a pharmaceutical gel of claim 19 wherein the silver carbene complex is selected from the group consisting of:

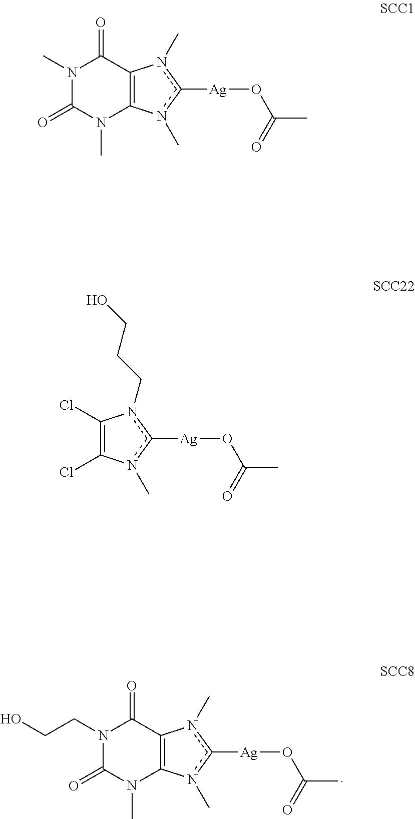

21. The method of forming a pharmaceutical gel of claim 19 comprising the additional step of mixing an additional drug component with one or both of the glutathione and silver carbene complex, the additional drug component selected from the group consisting of quinolone compounds and derivatives thereof; fluoroquinolone compounds and derivatives thereof; penicillin compounds and derivatives thereof; aminoglycoside compounds and derivatives thereof; cephalosporins compounds and derivatives thereof; glycopeptides and derivatives thereof; sulfonamides and derivatives thereof; tetracycline and derivatives thereof; steroids and derivatives thereof; anti-inflammatory compounds and derivatives thereof; analgesic compounds and derivatives thereof; anti-fungal compounds and derivatives thereof; anti-bacterial compounds and derivatives thereof; and tissue growth factors.

22. The method of claim 19, wherein said steps of mixing and allowing are carried out at room temperature and pressure.
